(19) United States
(12) Patent Application Publication
(10) Pub. No.: US 2005/0216026 A1
(43) Pub. Date: Sep. 29, 2005

(54) Guidance system for spinal stabilization

(76) Inventor: Brad S. Culbert, Rancho Santa Margarita, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/036,781

(22) Filed: Jan. 14, 2005

Related U.S. Application Data
(60) Provisional application No. 60/536,442, filed on Jan. 14, 2004.

(51) Int. Cl. A61B 17/56

(52) U.S. Cl. 606/96; 606/130

(57) ABSTRACT

A guidance system comprises a variably positioned components that assist a user in obtaining a desired alignment (e.g., insertion position and angle) with respect to a patient. Once the desired alignment is obtained, the system may be locked into place or allowed to float depending on user preferences. In one embodiment, the system is configured for use in spinal fixation applications.

### PRIORITY INFORMATION

This application claims the benefit of U.S. Provisional Application No. 60/536,442, filed January 14, 2004.

### BACKGROUND OF THE INVENTION

1. Field of the Invention

The present invention relates to medical devices and, more particularly, to methods and apparatus for spinal stabilization.

2. Description of the Related Art

The human spine is a flexible weight bearing column formed from a plurality of bones called vertebrae. There are thirty three vertebrae, which can be grouped into one of five regions (cervical, dorsal, lumbar, sacral, and coccygeal). Moving down the spice, there are generally seven cervical vertebra, twelve dorsal vertebra, five lumbar vertebra, five sacral vertebra, and four coccygeal vertebra. The vertebra of the cervical, dorsal, and lumbar regions of the spine are typically separate throughout the life of an individual. In contrast, the vertebra of the sacral and coccygeal regions in an adult are fused to form two bones, the five sacral vertebra which into extend the formation of the sacrum and the four coccygeal vertebra which into the coccyx.

In general, each vertebra contains an anterior, solid segment or body and a posterior segment or arch. The arch is generally formed of two pedicles and two laminae, supporting seven processes—four articular, two transverse, and one spinous. There are exceptions to these general characteristics of a vertebra. For example, the first cervical vertebra (atlas vertebra) has neither a body nor spinous process. Also, the second cervical vertebra (axis vertebra) has an odontoid process, which is a strong, prominent process, shaped like a tooth, rising perpendicularly from the upper surface of the body of the axis vertebra. Further details regarding the construction of the spine may be found in such common references as Gray's Anatomy, Crown Publishers, Inc., 1977, pp. 33-54, which is herein incorporated by reference.

The human vertebrae and associated connective elements are subjected to a variety of diseases and conditions which cause pain and disability. Among these diseases and conditions are spondylosis, spondylolisthesis, vertebral instability, spinal stenosis and degenerated, herniated, or degenerated and herniated intervertebral discs. Additionally, the vertebrae and associated connective elements are subject to injuries, including fractures and torn ligaments and surgical manipulations, including laminectomies.

The pain and disability related to the diseases and conditions often result from the displacement of all or part of a vertebra from the remainder of the vertebral column. Over the past two decades, a variety of methods have been developed to restore the displaced vertebra to their normal position and to fix them within the vertebral column. Such methods typically include fixation systems that are used for the stabilization of fractures and/or fusion of various portions of the spine. These fixation systems may include a variety of longitudinal elements such as rods or plates which span two or more vertebra and are affixed to the vertebra by various fixation elements such as wires, staples, and screws (e.g., pedicle screws which are often inserted through the pedicles of the vertebra, See e.g., FIG. 1D). These systems may be affixed to either the posterior or the anterior side of the spine. Another type of fixation system utilizes facet screws for stabilization of the spine. Such facet screws may be used to secure two adjacent vertebrae to each other in a trans-laminar, trans-facet or trans-facet pedicle (e.g., Boucher technique applications). See e.g., FIGS. 1A-1C.

Because the outer surface of the vertebrae is typically non-planer and the structure of the vertebrae is relatively complex, it is important that the fixation elements (e.g., wires, staples and/or screws) are properly aligned when they are inserted into the vertebrae. Improper alignment may result in the fixation element extending improperly completely through a vertebrae and into the spinal column and/or the fixation element being positioned in an unstable area of the vertebrae. However, achieving and maintaining accurate positioning and guidance of these fixation elements has proven to be quite difficult in practice. Such positioning difficulties are further complicated by the fact that the alignment angle for a fixation device through one vertebral body or pair of vertebral bodies will be unique to that individual due to individual differences in the spinal curvature and anatomies etc.

Accordingly, there is a general need in the art for providing and improved surgical guidance system and method, and in particular, and improved surgical guidance system and method for spinal fixation.

### SUMMARY OF THE INVENTION

There is provided in accordance with one embodiment of the present invention, a guidance system comprising variably positioned components that assist the user in obtaining the desired alignment (e.g., insertion position and angle) with respect to the spine for various fixation devices (e.g., bone screws) into the spine. Once the desired alignment is obtained, the system may be locked into place or allowed to float depending on user preferences. In one embodiment, the system is configured for use in spinal fixation applications. In modified embodiments, the system may also be configured for other surgical procedures (e.g., bone fixation, fracture stabilization, etc.) that requiring accurate alignment for placement of various surgical devices (e.g., screws, wires, or other hardware). Other non-limiting applications include neurosurgery, cardiology, nephrology, etc.

In one embodiment, the system comprises a frame which may be attached to an operating room table if desired, or anchored in a variety of other ways during surgery. Preferably, the frame may be adjusted in a first direction (e.g., anterior-posterior with respect to the patient). The frame includes a moveable structure that is configured to permit translation of the moveable structure in a second and/or third direction (e.g., medial-lateral and superior-inferior directions). The moveable structure preferably also allows adjustment of the angle and/or trajectory in the plane defined by the second and third directions and/or a plane defined by the second and the first directions plane of the device. Once the desired position and angles are set, an additional guide may be introduced if necessary, or a guide wire may be introduced directly through the moveable structure.

In another embodiment, a guidance system is provided for use in a spinal fixation procedure. The system comprises a support member which can be positioned a defined distance in a first direction from a patient. A first moveable member is configured for movement along the support member in a second direction. A second moveable member is configured for movement along the second moveable member in a third direction. A tool guide is carried by the second moveable member. The tool guide is configured to support a tool and to allow movement of the tool such that a trajectory of the tool with respect to the patient may be adjusted within in a first plane defined by the second and third directions and second plane defined by the third and first directions as the first and second moveable members are moved along the second and third directions respectively.

Another embodiment of the invention comprises a method for aligning a tool with respect to a patient. The method comprises providing a tool guide. The tool guide is positioned in a coordinate system comprising a first, second and third direction. The tool guide is moveably positioned within a guidance system with respect to the second and third directions. The tool guide is also configured to allow the trajectory of a tool carried by the tool guide to be adjusted within in a first plane defined by the second and third directions and second plane defined by the third and first directions as the first and second moveable members are moved along the second and third directions respectively. A distal tip of the tool is positioned at a desired target point. A proximal end of the tool is adjusted to adjust the trajectory of the tool in either the first plane or the second plane while the tool guide moves with respect to the second and third directions. A fixation device is locked limit the movement of the tool guide with respect to the second and third directions once the desired trajectory is achieved.

Another embodiment of the present invention comprises a method for aligning a tool with respect to a patient. The method comprises providing a tool guide. The tool guide is positioned in a coordinate system comprising a first, second and third direction. The tool guide is moveably positioned within a guidance system with respect to the second and third directions. The tool guide is also configured to allow the trajectory of a tool carried by the tool guide to be adjusted within in a first plane defined by the second and third directions and second plane defined by the third and first directions as the first and second moveable members are moved along the second and third directions respectively. A distal tip of the tool is positioned at a desired target point. A proximal end of the tool is adjusted to adjust the trajectory of the tool in either the first plane or the second plane while the tool guide moves with respect to the second and third direction. The trajectory of the tool in the first plane with respect to the patient is viewed with an imaging system. The position of the tool guide with respect to the second direction is fixed. The trajectory of the tool in the second plane is viewed with respect to the patient with an imaging system. The position of the tool guide is fixed with respect to the third direction.

Further features and advantages of the present invention will become apparent to those of skill in the art in view of the detailed description of preferred embodiments which follows, when considered together with the attached drawings and claims.

### BRIEF DESCRIPTION OF THE DRAWINGS

FIGS. 1A-1D illustrate various techniques for stabilizing the spine that utilize facet screws or pedicle screws.

FIG. 2 is a perspective view of an exemplary embodiment of a guidance system;

FIG. 2A is a closer view of a portion of the guidance system of FIG. 2;

FIG. 3 is a perspective view of a second embodiment of a guidance system;

FIG. 4 is a perspective view of third embodiment of a guidance system;

FIG. 5 is a perspective view of a fourth embodiment of a guidance system;

FIG. 6 is a closer view of a portion of the guidance system of FIG. 5;

FIG. 7 is a perspective view of fifth embodiment of a guidance system;

FIG. 8 is a closer view of a portion of the guidance system of FIG. 7;

FIG. 9 is a perspective view of a sixth embodiment of a guidance system; and

FIG. 10 is a perspective view of a seventh embodiment of a guidance system.

### DETAILED DESCRIPTION OF THE PREFERRED EMBODIMENTS

Although the exemplary embodiments of a guidance system and method will be disclosed primarily in the context of a spinal fixation procedure, the methods and structures disclosed herein may also find use in any of a variety medical applications, as will be apparent to those of skill in the art in view of the disclosure herein. For example, the methods and apparatus may be applicable to any of a variety of orthopedic procedures such as the fixation of proximal fractures of the femur and a wide variety of fractures and osteotomies, of the hand and non-orthopedic procedures.

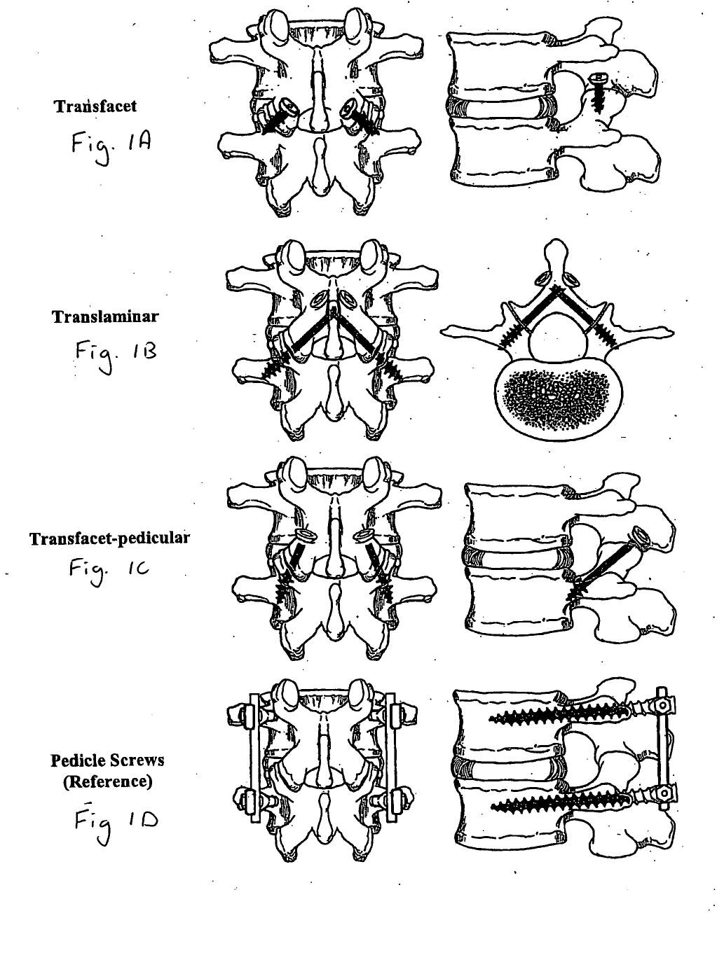

As mentioned above, the exemplary embodiments of the guidance system and method may be used to insert a bone fixation device that may be used in a variety of techniques to stabilize the spine. In such techniques, the bone fixation devices may be used as pedicle or facet screws that may be unilaterally or bilaterally symmetrically mounted on adjacent or non-adjacent vertebrae and used in combination one or more linkage rods or plates to facilitate fusion of one or more vertebrae. See e.g., FIG. 1D. In other techniques, the bone fixation devices may be used as a fixation screw to secure two adjacent vertebra to each other in a trans-laminar, trans-facet or trans-facet-pedicle (e.g., the Boucher technique) applications (see e.g., FIGS. 1A-1C). One of skill of the art will also recognize that the embodiments of the guidance system and method may also be used to insert bone fixation devices for posterior stability after laminectomy, artificial disc replacement, repairing odontoid fractures and other fractures of the spine, and other applications for providing temporary or permanent stability in the spinal column.

In one embodiment, the alignment or guidance system comprises variably positioned components that assist the user in obtaining the desired alignment (e.g., insertion position and angle) with respect to the spine for various fixation devices (e.g., bone screws) into the spine. Once the desired alignment is obtained, the system may be locked into place or allowed to float depending on user preferences. As mentioned above, in the exemplary embodiments, this system is configured for use in spinal fixation applications. In particular, the system may be used to align facet screws. In modified embodiments, the system may also be configured for other surgical procedures that requiring accurate alignment for placement of various surgical devices (e.g., screws, wires, or other hardware).

In one exemplary embodiment, the system comprises a frame which may be attached to an operating room table if desired, or anchored in a variety of other ways during surgery. Preferably, the frame may be adjusted in a first direction (e.g., anterior-posterior with respect to the patient). The frame includes a moveable structure that is configured to permit translation of the moveable structure in a second and/or third direction (e.g., medial-lateral and superior-inferior directions). The moveable structure preferably also allows adjustment of the angle and/or trajectory in the plane defined by the second and third directions and/or a plane defined by the second and the first directions plane of the device. Once the desired position and angles are set, an additional guide may be introduced if necessary, or a guide wire may be introduced directly through the moveable structure.

In one embodiment, the system may be used in combination with an imaging system, such as, for example, x-ray or fluoroscopy. The entire system size will vary depending on the particular procedure, but in one embodiment, the system is approximately 24″ wide by 12″ long to allow for full translation across an operating table, and a generous range along the patient.

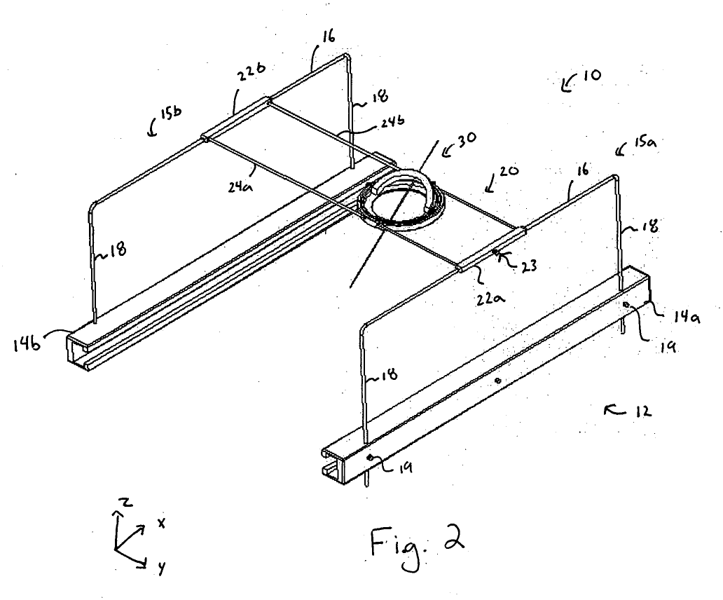

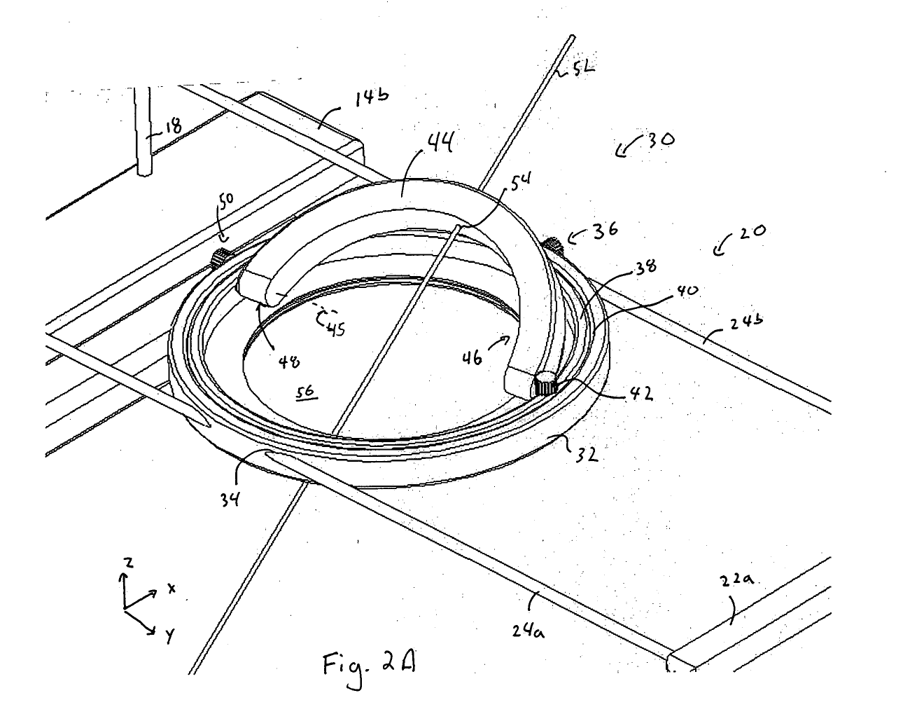

With reference now to FIG. 2, an exemplary embodiment of a guidance system 10 includes a base 12 comprising a pair of brackets 14a, 14b that may be secured to an operating room table (not shown) such that a patient may be positioned face down between the brackets 14a, 14b. The system 10 also includes a pair of vertical frames 15a, 15b. Each vertical frame 15a, 15b includes an x-direction rail 16 and a pair of vertical members 18. As shown in FIG. 2, in the exemplary embodiment, the x-direction rails 16 extend in substantially in the x-direction in an x-y plane while the vertical members 18 extend substantially in the z-direction in an x-z plane.

It should be noted that in this description of the exemplary embodiments, reference will be made to a traditional orthogonal three-dimensional coordinate system shown in FIG. 2. This coordinate system comprises three directions, which have been identified as the x, y and z directions, which are substantially orthogonal to each other. The x-y plane is positioned generally parallel to the operating room table and generally parallel the surgical site in most surgical applications. Correspondingly, the z-direction extends generally perpendicularly away from the surgical table and in most surgical applications in a vertical direction away from the surgical site. In the exemplary embodiments, various components will be described with reference to the directions and planes of this coordinate system. For example, such components may be described as extending in these directions, or lying or rotating within planes described by these directions. However, in modified embodiments, the coordinate system may be rotated or skewed with respect to the operating table and/or a non-traditional three dimensional coordination system (e.g., a system in which the x, y and z directions are not orthogonal to each other) may be used. Those of skill in the art will recognize in light of the disclosure herein that the exemplary embodiments may be adapted to correspond to such coordinate systems.

The vertical members 18 extend through the openings formed in the brackets 14a, 14b. In the exemplary embodiment, the position of the x-direction rails 16 in the z-direction (i.e. the height) with respect to the table may be adjusted depending on patient size or user preference by adjusting the position of the vertical member 18 within the bracket 14a, 14b. Various fixation devices 19 (e.g. set screws) may be used to secure the position of the vertical members 18 with respect to the brackets 14a, 14b.

A moveable frame 20 is positioned for movement along the x-direction rails 16. To facilitate such movement, in the illustrated embodiment, the moveable frame 20 includes a pair of moving members 22a, 22b that are configured to move along the x-directions rails 16 such that the moveable frame 20 is moveable in the x-direction. The moving members 22a, 22b may configured in any of variety forms to facilitate sliding movement along the x-direction rails 16. For example, in the illustrated embodiment, the moving members 22a, 22b comprise a U-shaped channel configured to fit over the respective x-direction rails 16. Ties or caps may be provided over the U-shaped channel to prevent the sliding members 22a, 22b from being dislodged from the x-direction rails 16 while still allowing the moving member 22a, 22b to slide along the x-direction rails 16. In other embodiments, the system 10 may be configured for non sliding movement by providing the device with rollers, pins, tracks, etc to facilitate movement along the x-direction rails.

With continued reference to FIG. 2, in the illustrated embodiment, the moveable frame includes a pair of y-direction rails 24a, 24b that extend substantially in the y-direction. The ends of the y-direction rails 24a, 24b are preferably coupled to the sliding members 22a, 22b. In this manner, the y-direction rails 24a, 24b may be moved in tandem in the x-direction along the x-direction rail of the frame. A fixation device 23, such as a set pin or screw, may be provided on one or both of the sliding members 22a, 22b to lock the position of the moveable frame 20 on the x-direction rails 16.

With reference now to FIG. 2A, a moveable tool guide 30 is configured for movement in the y-direction on the moveable frame 20 along the y-direction rails 24a, 24b. In the exemplary embodiment, the moveable tool guide 30 comprises a base member 32. The base member 32 includes a pair of bores 34 though which the y-direction rails 24a, 24b extend. In this manner, the tool guide 30 may move along the y-directions rails 24a, 24b in the y-directions. Of course in modified embodiments, the base member 30 may include a U-shape channel, wheels, pins, etc. or other suitable structure(s) for facilitating movement in the y-direction along the rails 24a, 24b. A fixation device 36 (e.g., a set screw) is preferably provided for locking the position of the moveable tool guide 30 on the y-direction rail 24a, 24b.

With continued reference to FIG. 2A, the moveable tool guide 30 includes a rotational member 38 that is configured to rotate with respect to the base member 32 in the x-y plane. In the illustrated embodiment, the base member 32 defines a circular channel 40 in which a rotational member 38 is positioned. In this manner, the rotational member 38 may be rotated within base member 32. The circular channel 40 and the rotational member 38 may be provided with intermeshing grooves and/or edges that are dimensioned such that the rotational member 28 may rotate freely within the base member 32. A fixation device 42 (e.g., a screw pin) may be provided to lock the position of the rotational member 38 with respect to the base member 32. As will be explained below, rotation of the rotational component 38 allows for angle adjustment in the x-y plane.

To provide for angle adjustment in the y-z plane, a pivoting member 44 is provided. The pivoting member is pivotably connected to the rotational component 38 such that the pivoting member 44 may be pivoted back and forth with respect to a pivot axis 45 coupled to the rotational component 38. In this manner, the pivoting member 44 may rotate with respect to the rotational component 38 and the base member 32. In the illustrated embodiment, the pivoting member 44 comprises an arced member 42 that is attached to the rotating member with two pivots (only one shown in FIG. 2) such that the pivoting member 44 may be pivoted with respect to the rotating member 38. A set screw or other fixation device 50 may be provided to lock the angular position of the pivoting member 44 with respect to the rotational member 38.

With continued reference to FIG. 2, a guide wire 52 may extend through an opening 54 in the pivoting member 44 and through an opening 56 in the rotational member 38. In a modified embodiment, an additional guide (e.g., an elongate tube or drill guide)or tissue protector may extend through the openings 54, 56.

In one embodiment of use, the vertical position (i.e., the z-direction) of the moveable frame 20 is adjusted with respect to the patient and/or the operating table by moving the vertical members 18 with respect to the brackets 14a, 14b. Once the moveable frame 20 is at the desired position with respect to the z-direction, the vertical members 18 may be secured within the brackets 14a, 14b by activating the fixation devices 19 on the brackets 14a, 14b.

The user then positions the distal tip of the guidewire 52 or additional guide at the proper entry point for the bone fixation device. In one exemplary embodiment, this may be the desired entry point on the facet of a particular vertebrae. With the distal tip of the guidewire 52 positioned at the desired location, the proximal end of the guidewire 52 may be adjusted so as to adjust the alignment of the guidewire 52 with respect to the x-y and y-z planes. As the proximal end is adjusted, the moveable frame 20 is free to move along the x-direction rail while the moveable tool guide 30 moves along the y-direction rail. Such movement of the proximal end while the distal end is fixed is facilitated by the rotational movement of the rotating member 38 and the pivoting movement of the pivoting member 44. In addition, the guidewire is preferably allowed to move longitudinally within the arced member 46 as the distance between the desired entry site and the moveable tool guide 30 is adjusted. Once the desired entry angle is achieved, the system 10 may be locked into place by activating the fixation devices 23, 32, 36, 50 on the moving member 22a, 22b, base member 32, rotational member 38, and/or pivoting member 44. For example, locking the system in the y-direction by activating the fixation device 36 on the base member 32, locks the angle in the y-z plane, while locking the system in the x direction by activating the fixation device 23 on the moving member 22a, 22b locks the angle in the x-y plane. The rotational and pivoting movement may also be secured by fixing the fixation devices 42, 50 for the rotation member 38 and pivoting member 44 to provide additional rigidity to the guidance system.

In one embodiment, the guidewire 52 may be used to puncture a hole through a vertebral body. In some embodiments, the hole may extend into an adjacent vertebral body. With the guidewire in position, a bone drill and/or fixation device (e.g., facet screw) may be inserted over the guidewire depending upon the clinical procedure.

This exemplary embodiment described above allows the user to place the tip of a guidewire, drill guide, or tissue protector at the point desired entry point on or inside the patient. With the desired entry point fixed, the proximal end of the guidewire, drill guide or tissue protector can be adjusted holding the entry point fix. When the desired entry alignment is achieved, the system can be locked to provide accurate and precise placement of the hardware.

In one embodiment, the guidance system may be used in combination with an imaging system, such as, for example, x-ray or fluoroscopy. In one embodiment of use, the distal end of the guidewire 52 may be positioned at the desired entry point on the bone. The imaging system may be used to provide a view of the patient in the x-y plane such that the surgeon may judge and adjust the alignment of the guidewire in the x-y plane. When the desired angle is achieved, the system 10 may be fixed in the x-y plane by locking the position of the fixation device 23 for the x-rails 16 to fix the position of the moveable tool guide 20 in the x-direction. The surgeon may then rotate the imaging device or use a second imaging device to view the patient in the z-y plane to judge and adjust the alignment of the guidewire 52 in this plane. Once the desired alignment is reached the system may be fixed by locking the fixation device 36 for the y-rail 24a, 24b to fix the position of the moveable tool guide 20 in the y-direction thereby fixing the alignment in the z-y plane. The previous steps may be repeated and/or their order reversed as desired by the surgeon. An imaging device in the z-y plane may also be used in other embodiments.

The above described system and method have several advantages. For example, the system 10 and method provides for a reduction in procedure time by simplifying the process of determining and fixing a proper entry angle for the fixation device. The device and methods are also intuitive to use. The device and methods may also be used with many percutaneous, minimally invasive procedures as well as open surgery procedures. The device 10 and method provide an infinite variability of entry angles.

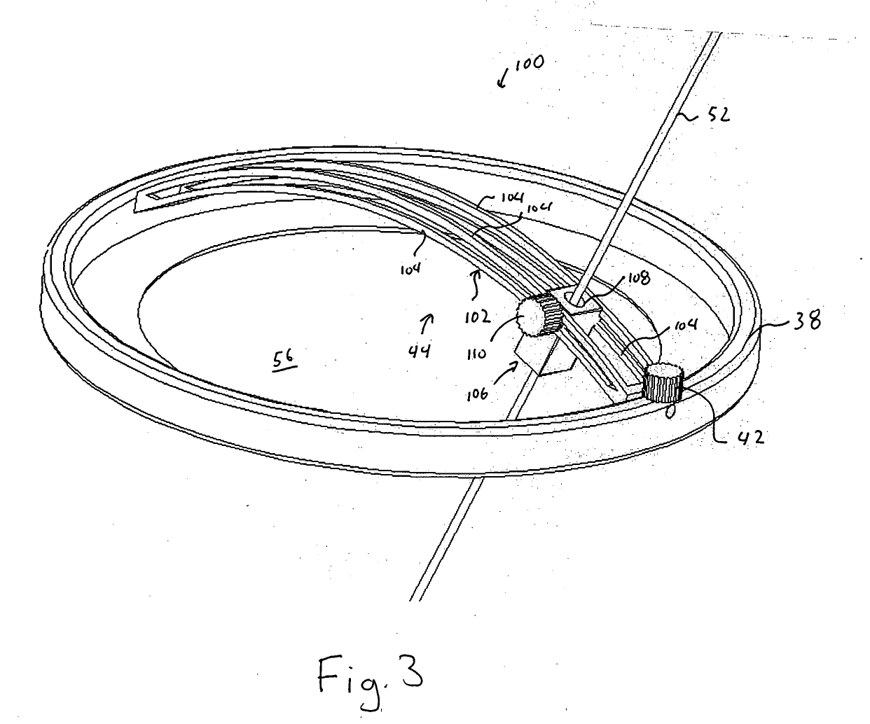

FIG. 3 illustrates another exemplary embodiment of a moveable tool component 100 that may be used within the moveable frame 20 described above. In this embodiment, the moveable tool 100 may include a base member (not shown) and rotational member 38 configured substantially as describe above. The pivoting member 44 in this embodiment comprises an arced sliding rail structure 102. The arced rail structure 102 comprises a plurality of arced rails 104 in which a sliding member 106 is positioned such that it can move along an arced path. The sliding member 106 is provided with a bore 108 through which the guide wire 52 or tool guide may extend. A fixation device 110 (e.g., a set screw) may extend through a gap between the arced rails 104 to secure the sliding member 106 at a particular position along the arc. Rotation of the rotational member 38 causes the arced sliding structure 104 to rotate allowing for alignment adjustment in the y-z plane, while movement of the sliding component 106 along the arced path allows for angle adjustment in the x-y plane.

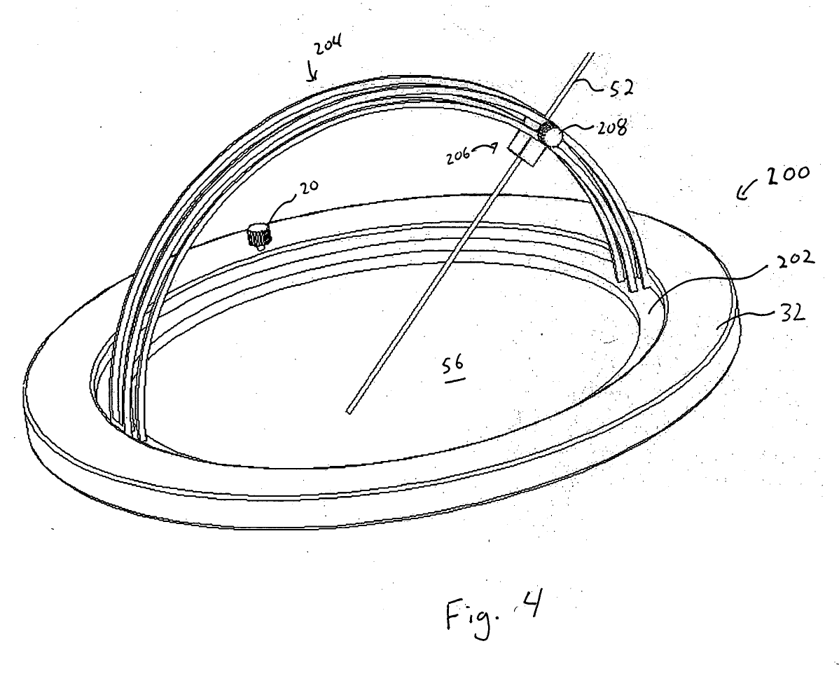

FIG. 4 illustrates another embodiment of a moveable tool component 200. This embodiment includes a base member 32 that may be configured as describes above. An inner component 202 is rotationally positioned within the base member 32. An arced sliding rail structure 204 similar to the arced rail structure describe above may be coupled to the inner component. In this embodiment, the base member 32 may move in the y-direction along the y-direction rails. The inner component 202 and the arced sliding rail structure 204 may rotate within the base member 32 for adjusting the angle in the x-y plane. A fixation device 205 (e.g., a set screw) may be provided for fixing the angle in the x-y plane. A slide component slides 206 within the arced sliding rail structure 204 to provide for angular adjustment in the y-z plane. A set screw or other type of lock 208 may be provided on the slide component 206 or arced structure 204 to set the desired position.

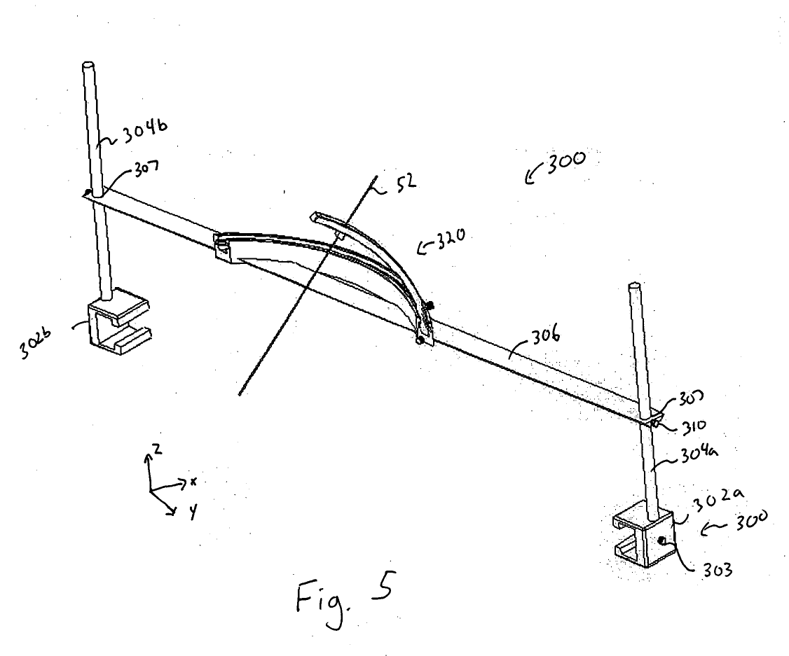

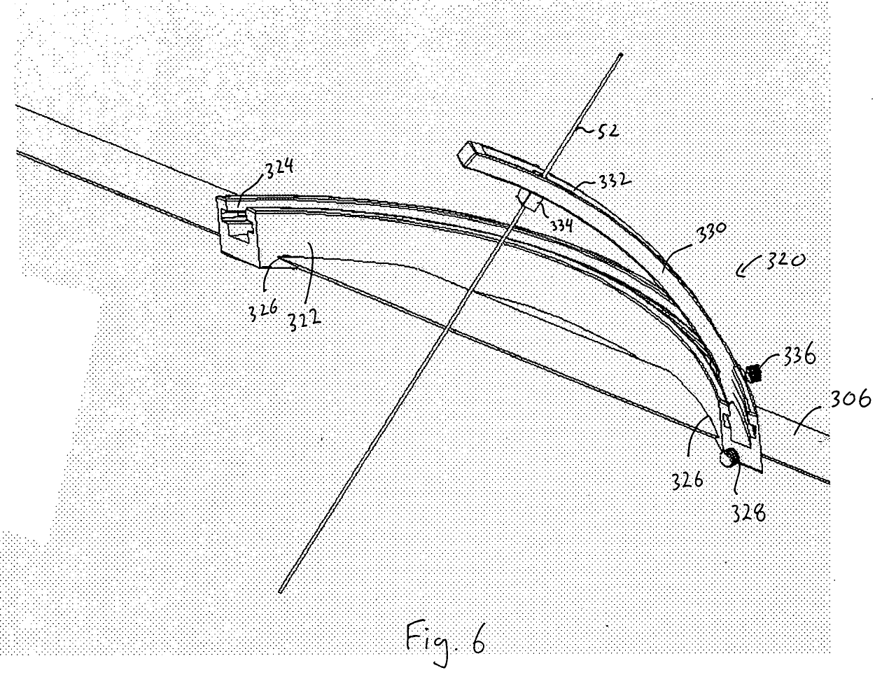

FIGS. 5 and 6 illustrate another modified embodiment of a guidance system 300. In this embodiment, a base 302 comprising a pair of brackets 302a, 302b that may be secured to an operating room table (not shown) such that a patient may be positioned face down between the brackets 302a, 302b. The system 300 also includes a two vertical members 304a, 304b that extend from the brackets 302a, 302b. A y-direction rail 306 extends between the vertical members 304a, 304b. In the exemplary embodiment, the vertical member 304a, 304b extends through an opening 307 in the y-direction rail 306. In this embodiment, the position of the y-direction member 306 in the z-direction (i.e. the height) with respect to the table may be adjusted depending on patient size or user preference by adjusting the position of the y-direction member 306 along the vertical members 304a, 304b. Various fixation devices 310 (e.g. set screws) maybe used to secure the position of the y-direction members 304a, 304b with respect to the vertical members 304a, 304b. In this embodiment, the brackets are used to adjust the position of the y-direction rail in the x-direction. For example, in one embodiment, the brackets are configured to slide along a rail on the operating table in the x-direction. A fixation device 303 (e.g., a set screw) may be used to fix the brackets 302a, 302b along such rails.

With particular reference to FIG. 6, a moveable tool component is positioned on the y-direction rail. In this embodiment, the moveable tool component 320 comprises an arced rail member 322. In the illustrated arrangement, the arced rail member 322 forms an arced U-shaped channel 324. A lower portion of the arced member includes a slot or opening 326 through which the y-direction 306 rail may extend. In this manner, the tool component 320 may slide back and forth on the y-direction rail 306 in the y-direction. A set screw or another type of suitable fixation device 328 is provided on the arced rail member 322 for securing the position of the arced rail member 322 on the y-direction rail 306.

A second arced rail member 330 is configured to slide within the U-shaped channel 324 of the first arced rail member 322. The second arced rail member 330 defines a channel 332 in which a sliding component 334 may be positioned. The sliding component 334 includes a bore or opening (not shown) through which the guidewire 52 or other suitable tool may extend. The proximal end of the second arced rail member 330 is slidably positioned within the channel 324 of the first arced member 322. A set screw or other suitable fixation device 336 may be provided for securing the position of the second arced rail member 330 on the first arced rail member 332. In a similar, manner the sliding component 334 may also include a set screw or other suitable fixation device (not shown) for securing its position on the second arced member 330.

In this embodiment, the base brackets 302a, 302b may be used for engaging the operating room table as described above and may also be used to provide for adjustability in the x-direction. The y-direction rail 306 may be moved along the vertical members 304a, 304b to provided adjustability in the z-direction. In this embodiment, the first arced rail member 322 provides for angle adjustment for in the x-y plane and the second arced rail component 330 provides for angle adjustment for the y-z plane. In this embodiment, predetermined lengths for the guide wire 52 (or guide) may be utilized and used to determine the radii of the first and second arced rail components 322, 330. In this manner, the system 300 may provide a constant center point about which any adjustments in angles are made.

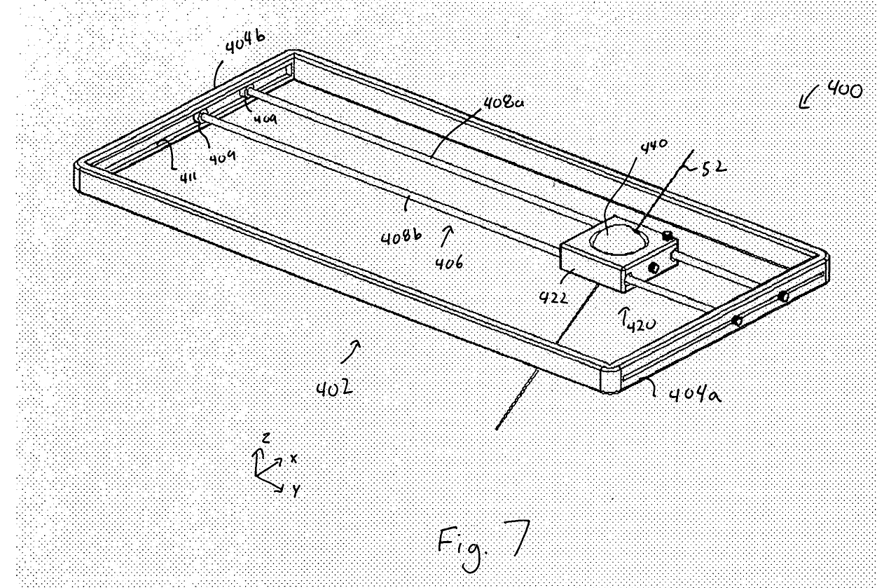

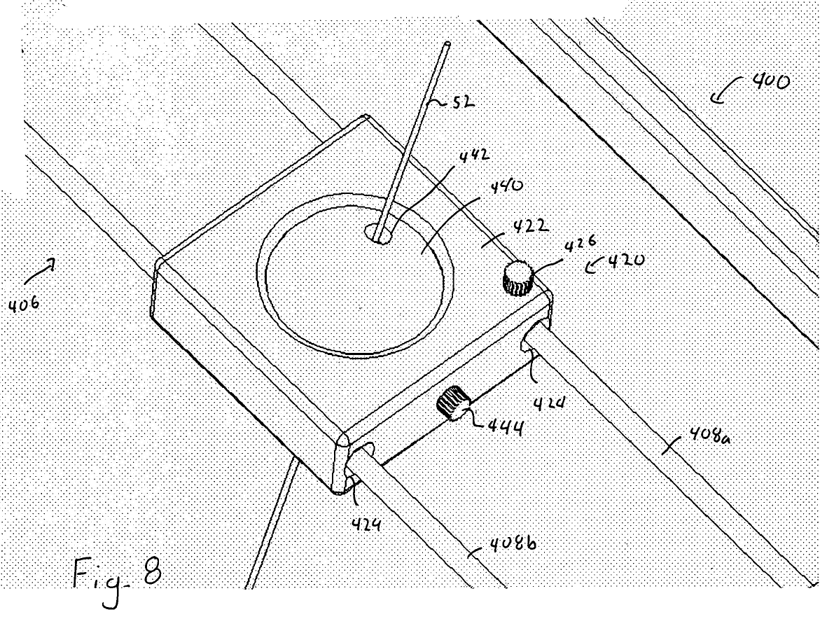

FIGS. 7 and 8 illustrate another embodiment of an exemplary guidance system 400. This embodiment includes a rectangular frame 402, which defines a pair of x-directions rails 404a, 404b. A moveable frame 406 is position within the rectangular frame 402. The moveable frame 406 includes a pair of y-direction rails 408a, 408b, which are configured to move in tandem along the x-direction rails 404a, 404b of the rectangular frame 402. In this embodiment, the ends of the y-direction rails 408a, 408b are provided with rollers 409, which move within channels 411 provided within the x-direction rails 404a, 404b. Of course, modified embodiments may use other components (e.g., linkages, sliding members, etc.) for facilitating such movement. Although not illustrated the rectangular frame 402 may be connected to the operating table by a secondary frame that provides for adjustment in the z-direction.

A moveable tool component 420 is positioned within the moveable frame 402. As shown in FIG. 8, the moveable tool component 420 includes a base member 422, which is moveable along the y-direction rails 408a, 408b of the moveable frame 402. In this embodiment, the base member 422 includes a pair of openings 424 through which the y-direction rails 408a, 408b extend such that the base member may slide along the y-direction rails. A fixation device 426 may be used to lock the position of the base member 422 with respect to the y-direction rails 408a, 408b.

A spherical rotational member 440 (e.g., a ball) is journalled for rotation within the base member 422. An opening 442 is provided in the rotational member 440 through which a guidewire 52 or other suitable tool extends. The spherical rotational member 440 allows for angle adjustment in both the x-y and y-z planes. By locking the moveable tool component 422 in the y direction on the y-direction rails, the angle in the y-z plane becomes locked, while locking the system 400 in the x direction along the x-direction rails 404a, 404b locks the angle in the x-y plane. Rotational movement of the rotational member 442 may be locked by a set screw 444 in the base member 422.

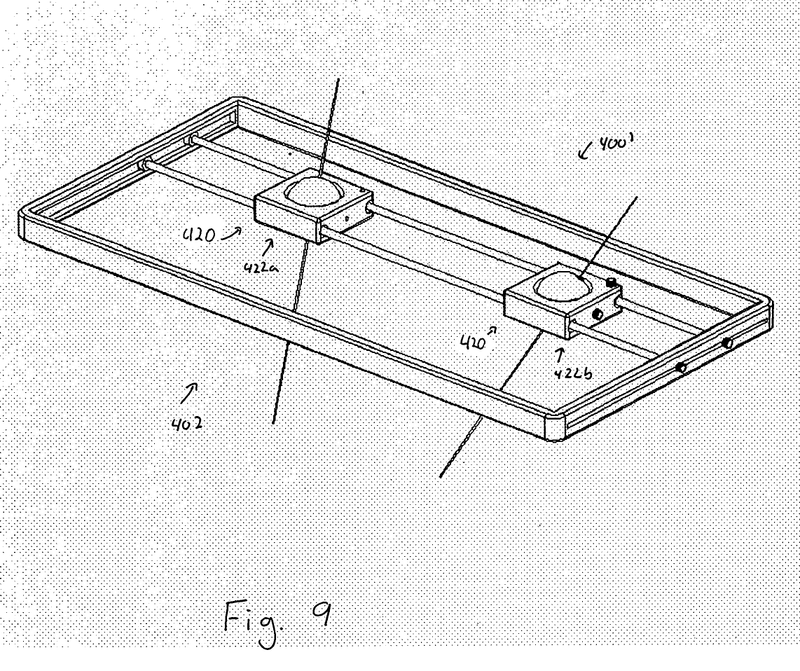

FIG. 9 illustrates another embodiment of a guidance system 400′ which is similar to the guidance system 400 described above with reference to FIGS. 7 and 8. In this embodiment, the system 400′ includes two moveable tool components 420a, 420b that may be arranged as described above. Both components 420a, 420b moveable along the y-direction rails 408a, 408b. This embodiment may be advantageous for procedures that require bilateral symmetry.

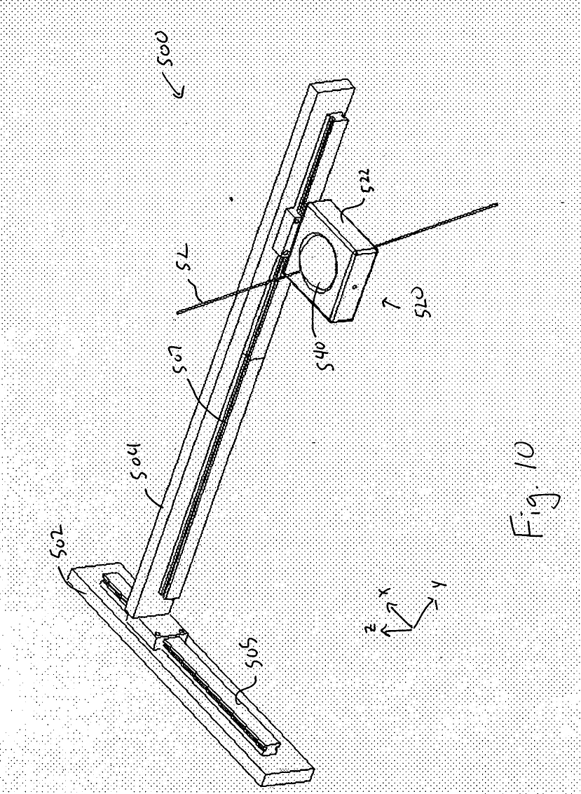

FIG. 10 illustrates another exemplary embodiment of a guidance system 500. This system is similar to the previous two embodiments in that it includes a tool component 520 comprising a base member 522 and a spherical rotational member 540 that allows for angle adjustment in both the x-y and y-z planes. For movement in the x and y directions, the system 500 include a x-direction slide rail 502 on which a y-direction rail 504 is moveably mounted. In this embodiment, an end of the y-direction rail 504 slides along a U-shaped rail 505. The base member 522, in turn, is slideably mounted on the y-direction member 504. In this embodiment, the base member 522 slides along a U-shaped rail 507 on the y-direction rail member 504. Movement in the z direction may be provided by adding any number of attachments between, for example, the surgical table or other support methods and the x-direction rail 502.

It should be appreciated that in the embodiments described above any a variety of linear motion components may be used to provide for the motion in the first, second and third directions (e.g., the x, y and z directions). Non-limiting examples of such linear motion components include any of a variety of sliding members, rail systems, tracks, and/or rollers. In a similar manner, in the embodiments described above, any of a variety of structures may be provided for providing rotation in the x-y, y-z and/or z-y planes. Non-limiting examples of such structures include various combinations and sub-combinations of arced guides, pivoting members, spherical rotational members, and/or circular rotational members. Linear and rotational movement in the various components may be locked with any of a variety of fixation devices, such as, for example, set screws, set pins, locks, ratchet structures etc.

Various materials may be used in the above described embodiments including plastic or metallic materials. Preferably, components of the system that may cause shadows during X-ray or other radiographic visualization methods would be manufactured from radiolucent materials as to prevent any visual obstruction of the desired location during the procedure.

As mentioned above, a bone fixation device may be inserted over the guidewire in a spinal fixation procedure. A preferred of such a bone fixation device is described in U.S. patent application Ser. No. 10/623,193, filed Jul. 18, 2003, which is hereby incorporated by reference herein and bodily incorporated into this application.

The specific dimensions of any of the components of the present invention can be readily varied depending upon the intended application, as will be apparent to those of skill in the art in view of the disclosure herein. Moreover, although the present invention has been described in terms of certain preferred embodiments, other embodiments of the invention including variations in dimensions, configuration and materials will be apparent to those of skill in the art in view of the disclosure herein. In addition, all features discussed in connection with any one embodiment herein can be readily adapted for use in other embodiments herein. The use of different terms or reference numerals for similar features in different embodiments does not imply differences other than those which may be expressly set forth. Accordingly, the present invention is intended to be described solely by reference to the appended claims, and not limited to the preferred embodiments disclosed herein.

1. A guidance system for use in a spinal fixation procedure, the system comprising:
a support member which can be positioned a defined distance in a first direction from a patient;
a first moveable member configured for movement along the support member in a second direction;
a second moveable member configured for movement along the second moveable member in a third direction; and
a tool guide carried by the second moveable member, the tool guide configured to support a tool and to allow movement of the tool such that a trajectory of the tool with respect to the patient may be adjusted within in a first plane defined by the second and third directions and second plane defined by the third and first directions as the first and second moveable members are moved along the second and third directions respectively.

2. The system as in claim 1, wherein the support member is configured to be coupled to an operating room table.

3. The system as in claim 2, wherein the support member is moveable with respect to the first direction.

4. The system as in claim 1, wherein the first direction corresponds to an anterior-posterior direction with respect to the patient; the second direction corresponds to a superior-inferior direction with respect to the patient and the third direction corresponds to a medial-lateral direction of the patient.

5. The system as in claim 1, wherein the tool comprises a guidewire.

6. The system as in claim 1, comprising a fixation device to fix the position of the first moveable member with respect to the frame.

7. The system as in claim 6, comprising a second fixation device to fix the position of the second moveable member with respect to the first moveable member.

8. A method for aligning a tool with respect to a patient, the method comprising:
providing a tool guide, the tool guide being positioned in a coordinate system comprising a first, second and third direction, the tool guide moveably positioned within a guidance system with respect to the second and third directions, the tool guide also being configured to allow the trajectory of a tool carried by the tool guide to be adjusted within in a first plane defined by the second and third directions and second plane defined by the third and first directions as the first and second moveable members are moved along the second and third directions respectively;
positioning a distal tip of the tool at a desired target point;
adjusting a proximal end of the tool to adjust the trajectory of the tool in either the first plane or the second plane while the tool guide moves with respect to the second and third directions; and
locking a fixation device to limit the movement of the tool guide with respect to the second and third directions once the desired trajectory is achieved.

9. The method as in claim 8, comprising rotating at least a portion of the tool guide as the trajectory of the tool is adjusted.

10. The method as in claim 9, comprising locking a second fixation device to limit the rotational movement of the tool guide.

11. The method as in claim 9, comprising adjusting the position of the tool guide with respect to the first direction.

12. The method of claim 8, wherein the tool comprises a guidewire and further comprising advancing a fixation device over the guidewire.

13. The method of claim 8, comprising positioning the tool guide over a patient's spine.

14. The method of claim 13, comprising advancing the tool into a portion of the spine.

15. The method as in claim 14, further comprising positioning the tip of the tool on a facet of a vertebrae.

16. The method as in claim 8, wherein the distal tip of the tool is kept fixed against the target point as the proximal end of the tip is adjusted.

17. A method for aligning a tool with respect to a patient, the method comprising:
providing a tool guide, the tool guide being positioned in a coordinate system comprising a first, second and third direction, the tool guide moveably positioned within a guidance system with respect to the second and third directions, the tool guide also being configured to allow the trajectory of a tool carried by the tool guide to be adjusted within in a first plane defined by the second and third directions and second plane defined by the third and first directions as the first and second moveable members are moved along the first and second directions respectively;
positioning a distal tip of the tool at a desired target point;
adjusting a proximal end of the tool to adjust the trajectory of the tool in either the first plane or the second plane while the tool guide moves with respect to the second and third directions;
viewing with the trajectory of the tool in the first plane with respect to the patient with an imaging system;
fixing the position of the tool guide with respect to the second direction;
viewing with the trajectory of the tool in the second plane with respect to the patient with an imaging system; and
fixing the position of the tool guide with respect to the third direction.

18. The method as in claim 17, wherein the distal tip of the tool is kept fixed against the target point as the proximal end of the tip is adjusted.
